(19) United States
(12) Patent Application Publication
(10) Pub. No.: US 2021/0310226 A1
(43) Pub. Date: Oct. 7, 2021

(54) AUTOMATED AND CORDLESS BIDET TOILET SEAT

(71) Applicant: Royce Newcomb, Fresno, CA (US)

(72) Inventor: Royce Newcomb, Fresno, CA (US)

(21) Appl. No.: 17/222,426

(22) Filed: Apr. 5, 2021

Related U.S. Application Data
(60) Provisional application No. 63/004,945, filed on Apr. 3, 2020.

(51) Int. Cl. E03D 9/00; E03D 9/08; A61L 9/14

(52) U.S. Cl. CPC E03D 9/007; E03D 9/08; A61L 2209/134; A61L 2209/11; A61L 2209/133; A61L 9/14

(57) ABSTRACT

One embodiment provides a bidet toilet seat system that includes at least one controller for controlling operations and features of the bidet toilet seat system. The system further includes a first water mixing tank including an air freshener compartment and first air freshener emitter. A second water mixing tank is attached with the first water mixing tank, and the second water tank includes a second air freshener emitter. A main bidet portion is integrated with a rechargeable power source that provides power for the at least one controller and the bidet toilet seat system, and with a main bidet water supply tank. The system further includes a toilet seat and toilet seat lid that are both connected to the main bidet portion.

### CROSS-REFERENCE TO RELATED APPLICATIONS

This application claims priority to the U.S. provisional patent application Ser. No. 63/004,945 filed on Apr. 3, 2020, entitled “DONE RIGHT BIDET,” which is incorporated herein by reference in its entirety.

### FIELD

The present embodiments relate to bidets, and in more particular, to automated and cordless bidet toilet seats for commercial and residential use.

### BACKGROUND

Bidets may be purchased as stand-alone systems. These systems need separate plumbing and their own space for installation, which many residential uses do not provide; besides the expense of installation and retrofitting homes, apartments and commercial bathrooms.

Bidets may additionally be purchased as a retrofit for regular types of toilets. These types of bidets must be plugged in with a cord and also require a ground-fault circuit interrupter (GFCI), which adds to the expense of installation and may not even be feasible for preexisting commercial and residential setup.

### SUMMARY

One embodiment provides a bidet toilet seat system that includes at least one controller for controlling operations and features of the bidet toilet seat system. The system further includes a first water mixing tank including an air freshener compartment and first air freshener emitter. A second water mixing tank is attached with the first water mixing tank, and the second water tank includes a second air freshener emitter. A main bidet portion is integrated with a rechargeable power source that provides power for the at least one controller and the bidet toilet seat system, and with a main bidet water supply tank. The system further includes a toilet seat and toilet seat lid that are both connected to the main bidet portion.

Another embodiment provides a bidet toilet seat system that includes at least one controller for controlling operations and features of the bidet toilet seat system. The system further includes at least one water mixing tank including an air freshener compartment and at least one air freshener emitter. A main bidet housing is integrated with a rechargeable power source that provides power for the at least one controller and the bidet toilet seat system. The main bidet housing including a main bidet water supply tank. The system further includes a toilet seat and toilet seat lid that are both connected to the main bidet housing.

Yet another embodiment provides a bidet toilet seat system that includes at least one controller for controlling operations and features of the bidet toilet seat system. The system further includes at least one water mixing tank including an air freshener cartridge compartment and at least one air freshener emitter. An air freshener cartridge is removably connected with an inlet and outlet of the air freshener cartridge compartment. The at least one air freshener emitter is connected to an air freshener spray unit. A main bidet housing is integrated with at least one rechargeable and replaceable battery that provides power for the at least one controller and the bidet toilet seat system. The main bidet housing includes a main bidet water supply tank. The system further includes a toilet seat and toilet seat lid that are both connected to the main bidet housing.

These and other features, aspects and advantages of the embodiments will become understood with reference to the following description, appended claims and accompanying figures.

### BRIEF DESCRIPTION OF THE DRAWINGS

The accompanying figures, which are incorporated herein, form part of the specification and illustrate one or more embodiments. Together with the description, the figures further explain the principles of the embodiments and to enable a person skilled in the relevant arts to make and use the embodiments.

FIG. 1A shows a cordless and automated bidet installed on an example toilet shown with the toilet seat lid of the bidet in a down position, according to one embodiment.

FIG. 1B shows a cordless and automated bidet installed on an example toilet shown with the toilet seat lid of the bidet in a raised position, according to one embodiment.

FIG. 2 is a close-up view of the bidet shown in FIG. 1B, according to one embodiment.

FIG. 3 shows a top view of a bidet controller for the bidet shown in FIGS. 1A-B, according to one embodiment.

FIG. 4 shows the bidet of FIG. 1B with the toilet seat and the toilet seat lid in raised positions, according to one embodiment.

FIG. 5 shows a close-up view of an integrated night-light in the toilet seat of the bidet shown in FIG. 1B, according to one embodiment.

FIG. 6 shows a close-up view of a first air freshener tank portion with replaceable air freshener unit and emitter of the bidet shown in FIGS. 1A-B, according to one embodiment.

FIG. 7A shows a rear view of the bidet shown in FIGS. 1A-B, according to one embodiment.

FIG. 7B shows a close-up of the bidet shown in FIG. 7A, according to one embodiment.

FIGS. 8A-D show different view of the first air freshener tank portion with replaceable air freshener unit and emitter of the bidet shown in FIG. 6, according to some embodiments.

FIG. 9A shows a rear perspective view of a second air freshener tank portion and power source of the bidet shown in FIGS. 1A-B, according to some embodiments.

FIG. 9B shows a front perspective view of the second air freshener tank portion and power source of the bidet shown in FIG. 9A, according to some embodiments.

FIG. 10 shows removal of an exemplar rechargeable power source of the bidet shown in FIG. 9A, according to some embodiments.

FIG. 11 shows insertion of the exemplar rechargeable power source of the bidet shown in FIG. 10, according to some embodiments.

FIG. 12 shows the bidet of FIGS. 1A-B installed on various different types and styles of toilets, according to some embodiments.

Although the specific features of the embodiments are shown in some drawings and not in others. This is done for convenience only, as each feature may be combined with any or all the other features in accordance with the embodiments herein.

### DETAILED DESCRIPTION

In the following detailed description, a reference is made to the accompanying drawings that form a part hereof, and in which the specific embodiments that may be practiced is shown by way of illustration. These embodiments are described in enough detail to enable those skilled in the art to practice the embodiments and it is to be understood that the logical, mechanical, and other changes may be made without departing from the scope of the embodiments. The following detailed description is therefore not to be taken in a limiting sense.

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

The word “exemplary” is used herein to mean “serving as an example, instance, or illustration.” Any embodiment described herein as “exemplary” is not necessarily to be construed as preferred or advantageous over other embodiments. Likewise, the term “embodiments” does not require that all embodiments include the discussed feature, advantage or mode of operation.

Various terms are used to refer to particular system components. This document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct communication between two or more elements, whether or not those elements are in physical contact with one another. Thus, if a first device couples to a second device, that connection may be through a direct connection or through an indirect connection via other devices and connections. The coupling can occur through two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The term “controller” means any device, system or part thereof that controls at least one operation. Such a controller may be implemented in hardware or a combination of hardware and software and/or firmware. The functionality associated with any particular controller may be centralized or distributed, whether locally or remotely.

The terminology used herein is for the purpose of describing particular example embodiments only and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

The embodiments relate to toilet seat bidet systems that are configured for attachment to a toilet system having a base or bowl portion and a tank portion (whether integrated or separate). One embodiment provides a bidet toilet seat system that includes at least one controller for controlling operations and features of the bidet toilet seat system. The system further includes a first water mixing tank including an air freshener compartment and first air freshener emitter. A second water mixing tank is attached with the first water mixing tank, and the second water tank includes a second air freshener emitter. A main bidet portion is integrated with a rechargeable power source that provides power for the at least one controller and the bidet toilet seat system, and with a main bidet water supply tank. The system further includes a toilet seat and toilet seat lid that are both connected to the main bidet portion. Another embodiment provides a bidet toilet seat system that includes at least one controller for controlling operations and features of the bidet toilet seat system. The system further includes at least one water mixing tank including an air freshener compartment and at least one air freshener emitter. A main bidet housing is integrated with a rechargeable power source that provides power for the at least one controller and the bidet toilet seat system. The main bidet housing including a main bidet water supply tank. The system further includes a toilet seat and toilet seat lid that are both connected to the main bidet housing. Yet another embodiment provides a bidet toilet seat system that includes at least one controller for controlling operations and features of the bidet toilet seat system. The system further includes at least one water mixing tank including an air freshener cartridge compartment and at least one air freshener emitter. An air freshener cartridge is removably connected with an inlet and outlet of the air freshener cartridge compartment. The at least one air freshener emitter is connected to an air freshener spray unit. A main bidet housing is integrated with at least one rechargeable and replaceable battery that provides power for the at least one controller and the bidet toilet seat system. The main bidet housing includes a main bidet water supply tank. The system further includes a toilet seat and toilet seat lid that are both connected to the main bidet housing.

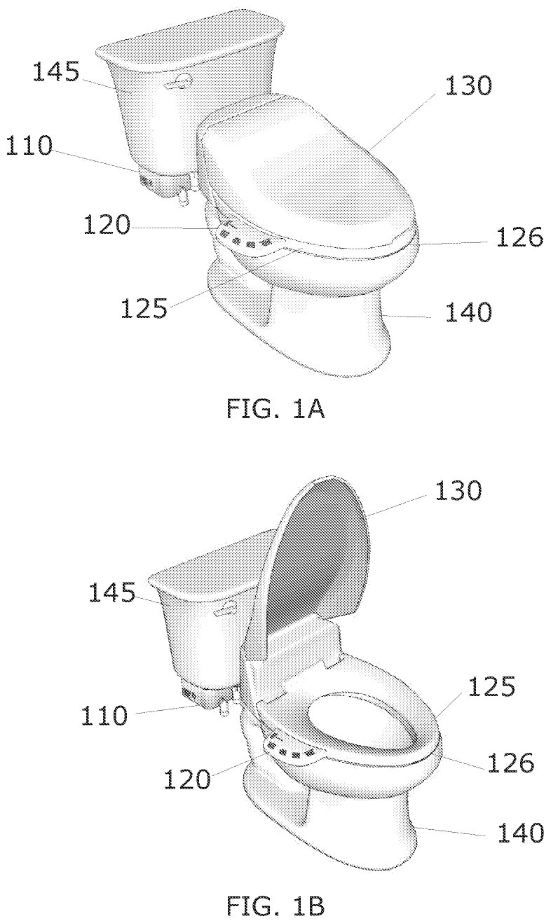

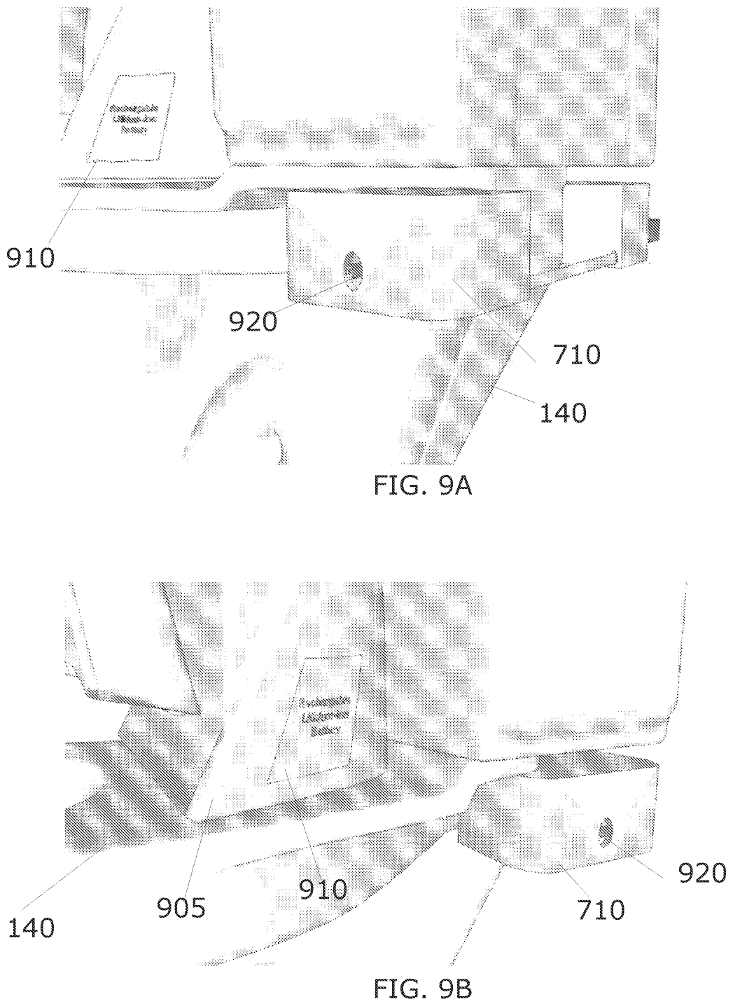

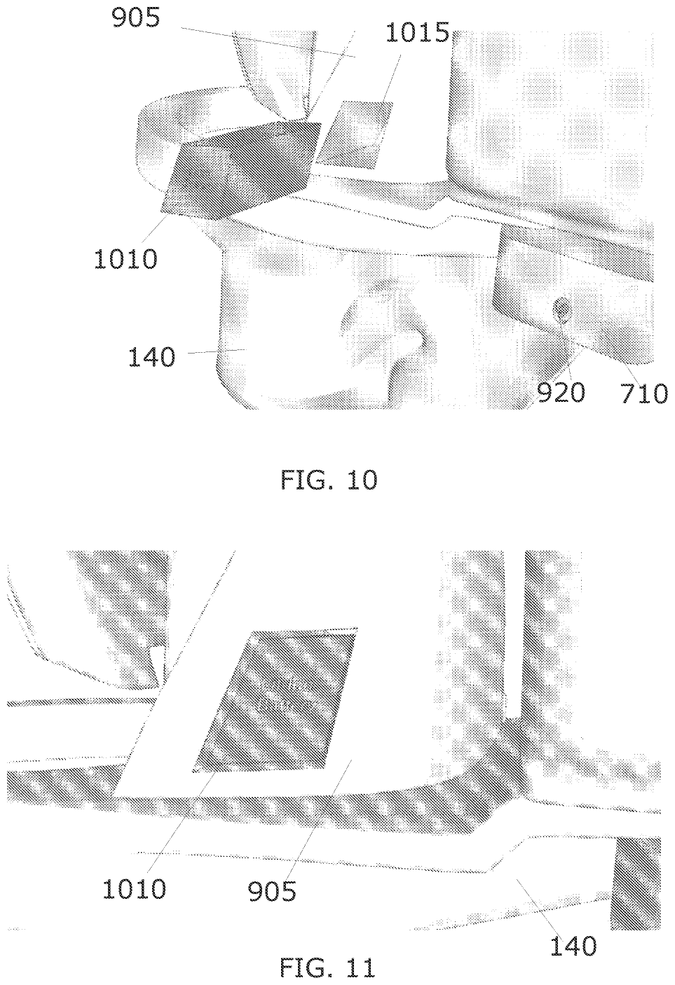

FIG. 1A shows a cordless and automated bidet installed on an example toilet shown with the toilet seat lid 130 of the bidet in a down position, according to one embodiment. As shown, the conventional toilet consists of the tank 145 and base 140. In some embodiments, the bidet includes the water tank 110 for anti-bacterial water-based air freshener dispersal from emitters 620 (FIG. 6) and 920 (FIGS. 9A-B), an integrated controller 120, a toilet seat 125 with integrated night-light 126, and toilet seat lid 130. Some embodiments relate to a multi-function style bidet toilet seat designed for residential and commercial properties that incorporates a rechargeable lithium-ion battery system with either a replaceable rechargeable battery 1010 (FIG. 10) or a wireless inductive charging system that does not require hardwired or cord connected electricity for power, and which dramatically reduces the cost of installation and increases the viability and market for these devices.

FIG. 1B shows the cordless and automated bidet installed on the example toilet shown with the toilet seat lid 130 of the bidet in a raised position, according to one embodiment. In some embodiments, the bidet may include features such as automatic night-lights via the night-light 126 (or additional lighting installed under the toilet seat 125 aimed within the toilet bowl, on the inner lower portion of the toilet seat 125, etc.), automatic seat raising and lowering (e.g., a button, a hand gesture sensed via a camera and software, etc., and a mechanism connected to the toilet seat 125 including gear(s) and motor, hydraulics, etc.), air freshening, that are integrated or designed into the bidet. In some embodiments, the bidet may include a built-in sensor that does not allow water to flow (e.g., using an electronic valve, check valve, etc.) in the bidet supply tube (connected to one or more nozzles within the toilet bowl) if the toilet seat 125 is not occupied (e.g., via sensors within the toilet seat 125, such as pressure or weight sensors, inductive touch sensors, heat sensors, etc.) to eliminate potential flooding and streaming water escaping the toilet bowl in case of malfunction.

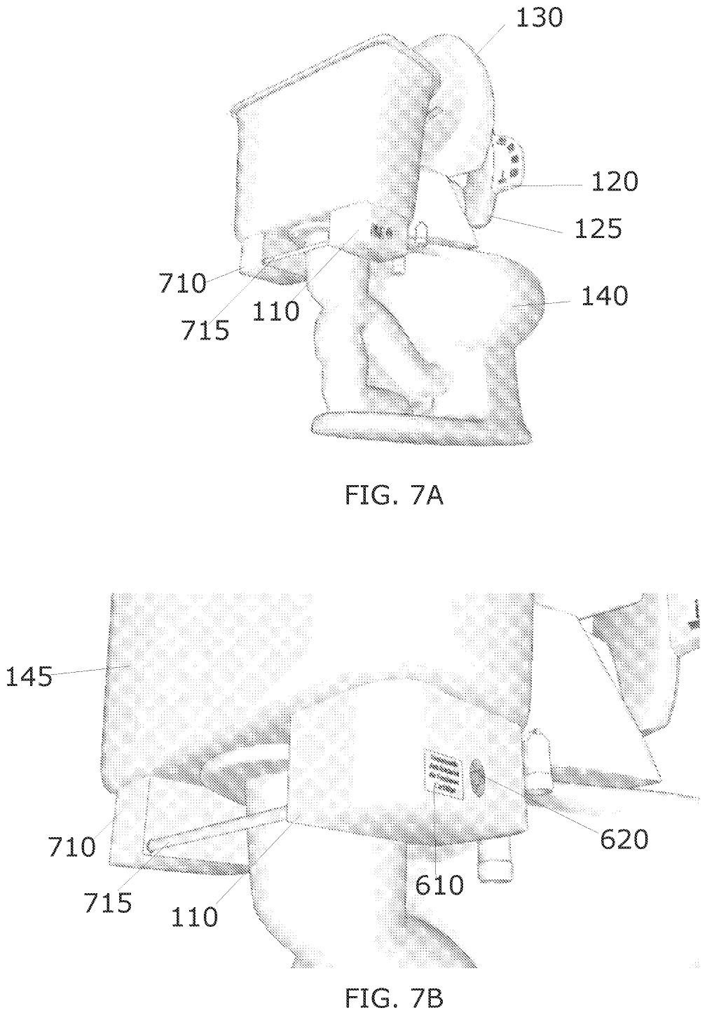

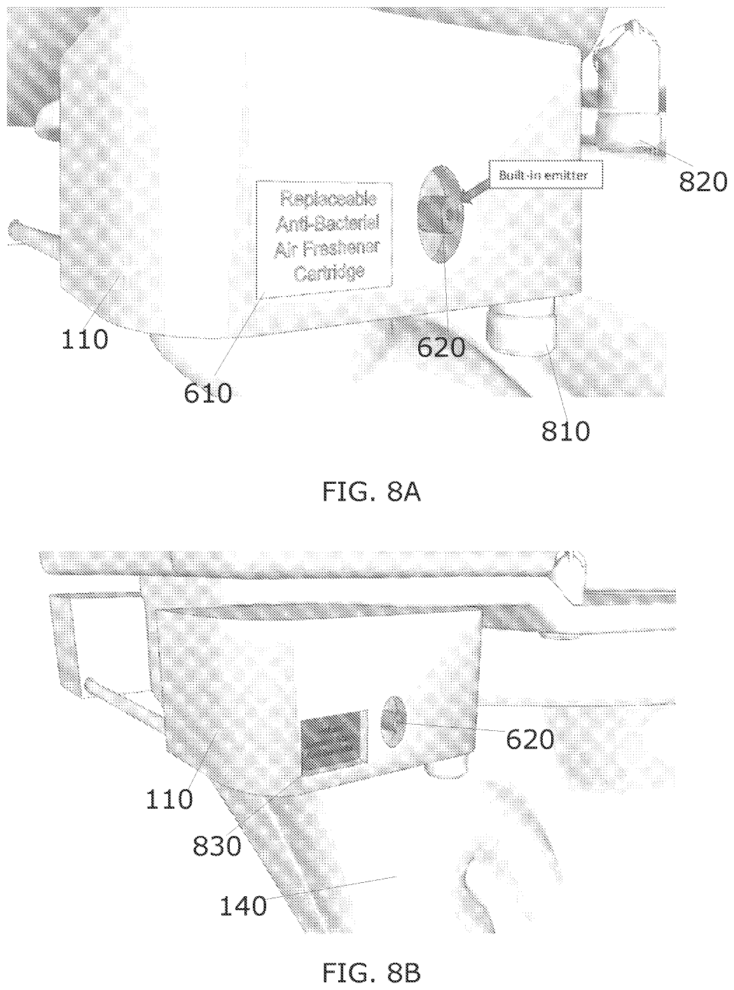

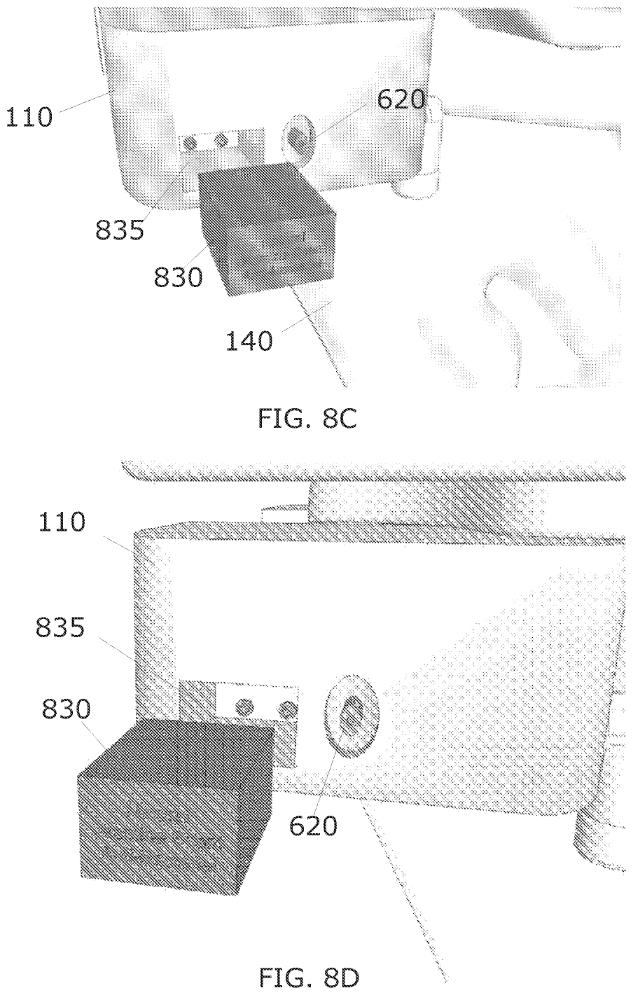

In one or more embodiments, the bidet may include the following additional features: heated toilet seat 125, selectable and temperature limited heated bidet water, a remote control with adjustable features for residential units, built-in anti-bacterial water-based air freshener with automatic side emitters 620 (FIG. 6) and 920 (FIGS. 9A-B) that are mounted under the tank 145 and that use a replaceable long-lasting powder pack 830 (FIGS. 8B-D) that is designed to easily mix with the water supply in the first water tank 110 and second water tank 710 (FIGS. 7A-B). In one or more embodiments, the features of the built-in anti-bacterial water-based air freshener of the bidet may contribute to the advantages of: having a non-aerosol air-freshener, low impact on sanitation, lower cost air freshener than conventional aerosol-based systems, anti-bacterial, kills germs, utilizes a built-in water supply, automatically sprays (e.g., after use, periodically timed, etc.), keeps a bathroom fresh at all times, etc.

In some embodiments, the features of the bidet may contribute to the advantages of: helping to minimize the use of toilet paper both in private residential and commercial applications, saving costs on the purchase of toilet paper while decreasing the ecological footprint, therefore producing a capital investment payback over time and potentially reducing global warming along with minimizing the impact to landfills and sewage systems.

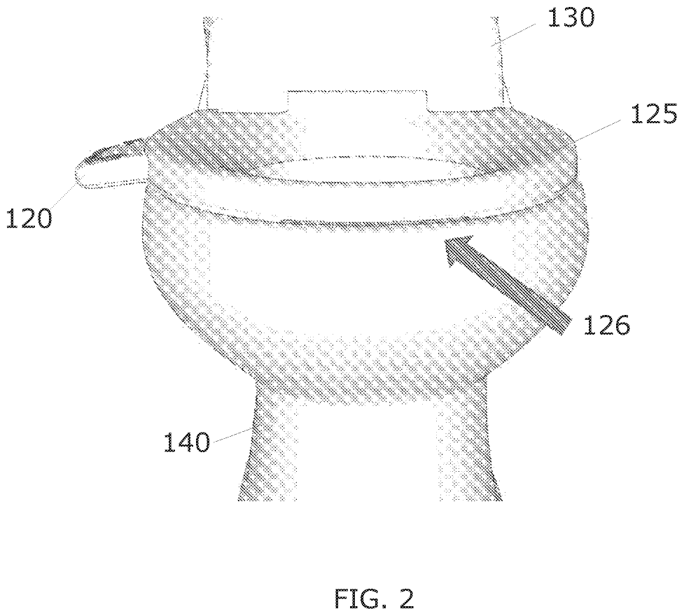

FIG. 2 is a close-up view of the bidet shown in FIG. 1B, according to one embodiment. In some embodiments, the integrated night-light 126 provides lighting in the toilet area such that additional lighting may be avoided, which helps save energy costs. Additionally, the night-light 126 may be enhanced with motion detection, dusk-to-dawn light sensing, dimming or set illumination settings, etc.

FIG. 3 shows a top view of a bidet controller 120 for the bidet shown in FIGS. 1A-B, according to one embodiment. In some embodiments, the controller 120 may include different functions, such as, but not limited to, water stream/spray direction, air drying blower/fan (e.g., simple room temperature, heat settings, automatic timing (e.g., 1 minute, 2 minutes, etc.), speed settings, etc.), toilet seat 125 heating (e.g., on/off, temperature settings, time of day settings, etc.), air freshener manual and automatic settings (e.g., timed directly after use, delayed after use, periodic settings, etc.), etc. In one embodiment, the controller 120 may include memory for different user preferences. In some embodiments, the controller 120 may include BLUETOOTH® functionality to be set using an app with a smart phone or device.

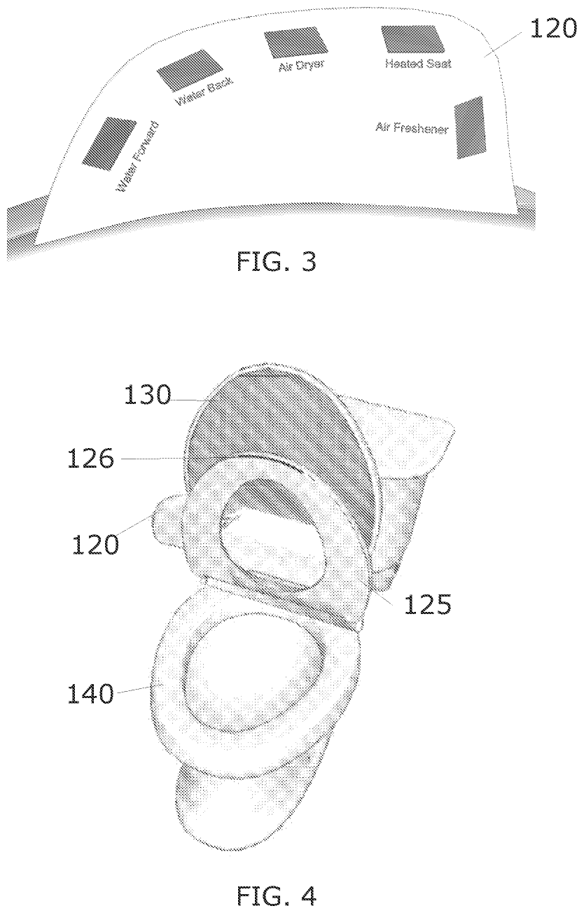

FIG. 4 shows the bidet of FIG. 1B with the toilet seat 125 and the toilet seat lid 130 in raised positions, according to one embodiment. In some embodiments, the toilet seat lid 130 may be manually lifted or automatically lifted. In one or more embodiments, the toilet seat lid 130 may automatically lift upon motion in front of the toilet for a particular duration (e.g., 2 sec., 3 sec., etc.), based on a gesture waving or moving over a sensor, based on natural language understanding (NLU) commands, facial recognition, artificial intelligence (AI) or machine learning (ML), etc. In some embodiments, the toilet seat 125 may automatically lift upon motion in front of the toilet for a particular duration (e.g., 2 sec., 3 sec., etc.), based on a gesture waving or moving over a sensor, based on NLU commands, facial recognition, AI or ML, a radio frequency identification (RFID) communication (e.g., an RFID fob or integrated remote controller and a transceiver integrated into the bidet system), etc. The automatic lifting contributes to the advantage of a non-touch system in commercial and residential environments. In some embodiments, the toilet seat 125 and the toilet seat lid 130 may automatically close or manual close using a soft-close mechanism.

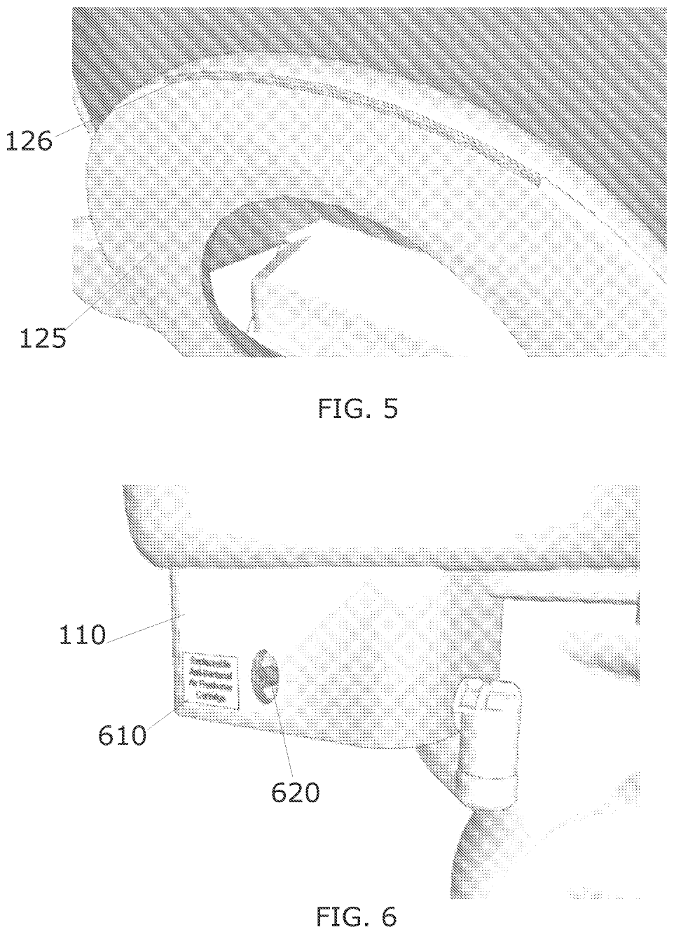

FIG. 5 shows a close-up view of an integrated night-light 126 in the toilet seat 125 of the bidet shown in FIGS. 1A-B, according to one embodiment. In one or more embodiments, the integrated night-light 126 is disposed on the lower outer peripheral of the toilet seat 125. In some embodiments, the night-light 126 may include light emitting diodes (LEDs), LED strip, etc. In some embodiments, the night-light 126 may include a particular solid color (e.g., blue, white, amber, etc.), a selectable color, sequencing, flashing (e.g., for notifications), etc. In one or more embodiments, the night-light 126 may include sound indications or a virtual voice to assist those with limited eye-sight.

FIG. 6 shows a close-up view of a first water tank 110 portion with replaceable air freshener unit 610 and emitter 620 of the bidet shown in FIGS. 1A-B, according to one embodiment. In some embodiments, the replaceable air freshener unit 610 includes an air freshener cover, which covers a compartment 835 (FIG. 8C) that holds a replaceable air freshener powder pack 830 (FIGS. 8B-D). In some embodiments, the emitter 620 provides an outlet for an air freshener and anti-bacterial emission or spray. In one or more embodiments, the emitter 620 may be fixed for a particular direction of emission, or selectable direction for emission (e.g., by turning the emitter, angling the emitter, etc.). In some embodiments, the bidet includes a water connector 810 (FIG. 8A) for the bathroom toilet water supply and a bidet water tank connector 820 (FIG. 8A) for the supply of water for the bidet. In some embodiments, a dual connector is attached to the toilet water supply line hose that connects to the toilet tank 145 (see, e.g., FIGS. 1A-B) and to the connector 820.

FIG. 7A shows a rear view of the bidet shown in FIGS. 1A-B, according to one embodiment. In some embodiments, the connecting tube 715 connects the first water tank 110 and the second water tank 710. The connecting tube 715 may be a fixed or replaceable solid polyvinylchloride (PVC) type of hose, a flexible hose, etc.

FIG. 7B shows a close-up of the bidet shown in FIG. 7A, according to one embodiment. In some embodiments, the connecting tube 715 may be replaced with a lower connecting tank (not shown) if a larger supply of water for the replaceable air freshener unit 610 is desired (e.g., commercial deployment).

FIGS. 8A-D show different view of the first water tank 110 portion with replaceable air freshener unit 610 and emitter 620 of the bidet shown in FIG. 6, according to some embodiments. In FIG. 8B, the replaceable long-lasting powder pack 830 is shown as inserted into the compartment 835 (FIG. 8C) that includes a water inlet and air freshener outlet connected to the emitter 620 (e.g., via a pump spray system). The built-in anti-bacterial water-based air freshener unit 610 with automatic side emitters 620 and 920 (FIG. 9A) mounts under the toilet tank 145. The replaceable long-lasting powder pack 830 is designed to easily mix with the water supply (e.g., OZMO anti-bacteria 1 powder, etc.). In some embodiments, the replaceable long-lasting powder pack 830 connects to the water supply, is designed to fit on most any toilet, and has electrical power from a rechargeable lithium-ion battery 1010 (FIG. 10) to spray automatically at timed intervals, manually, a set time after use of the bidet, etc. The replaceable long-lasting powder pack 830 is designed to be easy to change and is also long lasting (depending on use).

FIG. 9A shows a rear perspective view of a second water tank 710 portion and power source 910 of the bidet shown in FIGS. 1A-B, according to some embodiments. FIG. 9B shows a front perspective view of the second water tank 710 portion and power source 910 of the bidet shown in FIG. 9A, according to some embodiments. In some embodiments, the power source 910 is located within the portion of the bidet that also includes the bidet water tank 905. In some embodiments, the use of a rechargeable lithium-ion battery 1010 (FIG. 10) for cordless operation makes the bidet much easier to install and use (not only in residential applications), because the hassle and expense of running a permitted electrical outlet and GFCI adds significant cost to the install and also eliminates many potential applications (because they are simply not viable). In one or more embodiments, the bidet is easily installed in a large commercial market of hotels (individual rooms), restaurants, stadiums, commercial buildings, airports, and most anywhere that has a private or public style restroom where large quantities of toilet paper are consumed over time.

FIG. 10 shows removal of an exemplar rechargeable power source (rechargeable lithium-ion battery 1010) of the bidet shown in FIG. 9A, according to some embodiments. In some embodiments, the rechargeable lithium-ion battery 1010 is easy to replace in the compartment 1015 (after removing an enclosure or door) when needed with a low battery indicator LED light, sound indicator, etc. In one or more embodiments, an inductive charging system is included in the bidet and never needs to be changed.

FIG. 11 shows insertion of the exemplar rechargeable power source (rechargeable lithium-ion battery 1010) of the bidet shown in FIG. 10, according to some embodiments. In some embodiments, the rechargeable lithium-ion battery 1010 may be removed by pushing the rechargeable lithium-ion battery 1010 using a spring mechanism (or similar function component) that pops out the rechargeable lithium-ion battery 1010. Insertion of the rechargeable lithium-ion battery 1010 is easily performed by pushing the rechargeable lithium-ion battery 1010 into the compartment 1015 until the spring mechanism (or similar function component) accepts the rechargeable lithium-ion battery 1010 such that the enclosure or door may shut properly to conceal the rechargeable lithium-ion battery 1010.

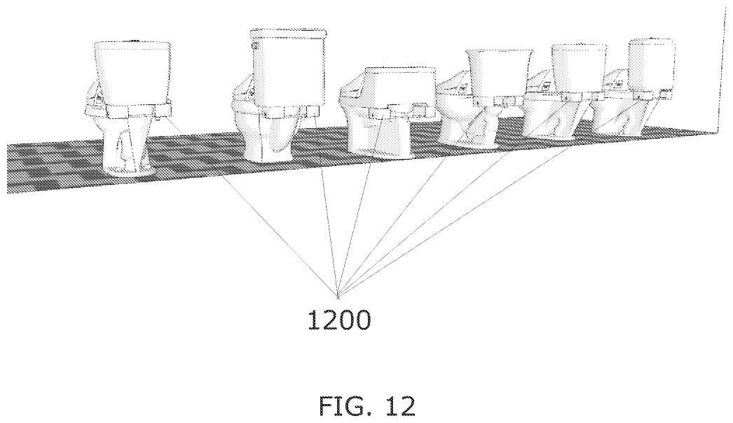

FIG. 12 shows examples 1200 of the bidet of FIGS. 1A-B installed on various different types and styles of toilets, according to some embodiments. One or more embodiments provide easy installation and there is no need to have electrical work performed (e.g., installation of a GFCI outlet, cutting walls, fixtures, etc. to install wiring, etc.). The bidet may also be installed on tankless toilet systems with an adapter to enable a water supply to supply water to the bidet.

References in the claims to an element in the singular is not intended to mean “one and only” unless explicitly so stated, but rather “one or more.” All structural and functional equivalents to the elements of the above-described exemplary embodiment that are currently known or later come to be known to those of ordinary skill in the art are intended to be encompassed by the present claims. No claim element herein is to be construed under the provisions of 35 U.S.C. section 112, sixth paragraph, unless the element is expressly recited using the phrase “means for” or “step for.”

While the foregoing written description of the embodiments enables one of ordinary skill to make and use what is considered presently to be the best mode thereof, those of ordinary skill will understand and appreciate the existence of variations, combinations, and equivalents of the specific embodiments, method, and examples herein. The embodiments should therefore not be limited by the above-described embodiments, method, and examples, but by all embodiments and methods within the scope and spirit as claimed.

What is claimed is:

1. A bidet toilet seat system comprising:
at least one controller for controlling operations and features of the bidet toilet seat system;
a first water mixing tank including an air freshener compartment and first air freshener emitter;
a second water mixing tank coupled with the first water mixing tank, the second water tank comprising a second air freshener emitter;
a main bidet portion integrated with a rechargeable power source that provides power for the at least one controller and the bidet toilet seat system, and with a main bidet water supply tank; and
a toilet seat and toilet seat lid that are both coupled to the main bidet portion.

2. The bidet toilet seat system of claim 1, wherein an air freshener cartridge is removably coupled with an inlet and outlet of the air freshener compartment.

3. The bidet toilet seat system of claim 2, wherein anti-bacterial air freshener powder from the anti-bacterial air freshener cartridge mixes with water from the first water mixing tank and the second water mixing tank.

4. The bidet toilet seat system of claim 3, wherein the first air freshener emitter is coupled with a first air freshener spray unit and the second air freshener emitter is coupled with a second air freshener spray unit.

5. The bidet toilet seat system of claim 3, wherein each of the first air freshener emitter and the second air freshener emitter emit an air freshener spray at one of a periodical time period, after a particular time period based on one or more sensors coupled to the toilet seat or manually based on an input received by the at least one controller.

6. The bidet toilet seat system of claim 1, wherein the rechargeable power source comprises at least one rechargeable and replaceable battery.

7. The bidet toilet seat system of claim 1, wherein the bidet toilet seat system is configured for attaching to a toilet having a base portion and a tank portion.

8. The bidet toilet seat system of claim 1, wherein the toilet seat is coupled with a night-light component on an outer perimeter.

9. The bidet toilet seat system of claim 1, wherein at least one of the toilet seat and the toilet seat lid are each configured to automatically raise up and down based on at least one of a voice command, a sensed motion, a sensed body position, or a touch input.

10. A bidet toilet seat system comprising:
at least one controller for controlling operations and features of the bidet toilet seat system;
at least one water mixing tank including an air freshener compartment and at least one air freshener emitter;
a main bidet housing integrated with a rechargeable power source that provides power for the at least one controller and the bidet toilet seat system, the main bidet housing including a main bidet water supply tank; and
a toilet seat and toilet seat lid that are both coupled to the main bidet housing.

11. The bidet toilet seat system of claim 10, wherein an air freshener cartridge is removably coupled with an inlet and outlet of the air freshener compartment.

12. The bidet toilet seat system of claim 11, wherein anti-bacterial air freshener powder from the anti-bacterial air freshener cartridge mixes with water from the at least one water mixing tank.

13. The bidet toilet seat system of claim 12, wherein the at least one air freshener emitter is coupled with an air freshener spray unit.

14. The bidet toilet seat system of claim 12, wherein the at least one first air freshener emitter emits an air freshener spray at one of a periodical time period, after a particular time period based on one or more sensors coupled to the toilet seat or manually based on an input received by the at least one controller.

15. The bidet toilet seat system of claim 10, wherein the rechargeable power source comprises at least one rechargeable and replaceable battery.

16. The bidet toilet seat system of claim 10, wherein the bidet toilet seat system is configured for attaching to a toilet having a base portion and a tank portion.

17. The bidet toilet seat system of claim 10, wherein the toilet seat is coupled with a night-light component on an outer lower perimeter.

18. The bidet toilet seat system of claim 10, wherein at least one of the toilet seat and the toilet seat lid are each configured to automatically raise up and down based on at least one of a voice command, a sensed motion, a sensed body position, or a touch input.

19. A bidet toilet seat system comprising:
at least one controller for controlling operations and features of the bidet toilet seat system;
at least one water mixing tank including an air freshener cartridge compartment and at least one air freshener emitter, wherein an air freshener cartridge is removably coupled with an inlet and outlet of the air freshener cartridge compartment, and the at least one air freshener emitter is coupled to an air freshener spray unit;
a main bidet housing integrated with at least one rechargeable and replaceable battery that provides power for the at least one controller and the bidet toilet seat system, the main bidet housing including a main bidet water supply tank; and
a toilet seat and toilet seat lid that are both coupled to the main bidet housing.

20. The bidet toilet seat system of claim 19, wherein:
anti-bacterial air freshener powder from the anti-bacterial air freshener cartridge mixes with water from the at least one water mixing tank;
the at least one first air freshener emitter emits an air freshener spray at one of a periodical time period, after a particular time period based on one or more sensors coupled to the toilet seat or manually based on an input received by the at least one controller;
the bidet toilet seat system is configured for attaching to a toilet having a base portion and a tank portion;
the toilet seat is coupled with a night-light component on an outer lower perimeter; and
at least one of the toilet seat and the toilet seat lid are each configured to automatically raise up and down based on at least one of a voice command, a sensed motion, a sensed body position, or a touch input.
